(19) United States
(12) Patent Application Publication
(10) Pub. No.: US 2014/0314883 A1
(43) Pub. Date: Oct. 23, 2014

(54) PROCESS FOR OBTAINING PARTIALLY PURIFIED EXTRACTS OF ANTIOXIDANT COMPOUNDS OF PALM FRUITS OF THE GENUS EUTERPE

(71) Applicants: AMAZON DREAMS INDUSTRIA E COMERCIO LTDA., Belem - Para (BR); UNIVERSIDADE FEDERAL DO PARA, Belem - Para (BR)

(72) Inventors: Hervé Louis Ghislan ROGEZ, Belem (BR); Braz Viana SARUBI NETO, Belem (BR); FabioÈ Gomes MOURA, Belem (BR)

(21) Appl. No.: 14/318,973

(22) Filed: Jun. 30, 2014

Related U.S. Application Data
(63) Continuation of application No. 13/814,186.

(30) Foreign Application Priority Data
Aug. 4, 2010 (BR) PI 1003060-3

(51) Int. Cl. A61K 36/889

(52) U.S. Cl. CPC A61K 36/889
USPC 424/727

(57) ABSTRACT

The present invention relates to a process for the partial and differential extraction that leads to obtaining extracts with appreciable amounts of antioxidants from fruits of palm trees of the genus Euterpe. The process comprises the steps of collecting the fruit, solid-liquid extraction (pulping 1) of the antioxidant compounds, softening and/or maceration of pulp and solid-liquid extraction (pulping 2 and/or 3) to obtain a standardized juice comprising both hydrophilic and lipophilic antioxidants, and an oily fraction. The process of the present invention also extends to a partial purification of the crude aqueous extract produced by the technique of adsorption on macroporous resins, resulting in higher purity extract of antioxidants. The extract or powder obtained after drying can be used in the food industry, cosmetics, pharmaceutical and herbal areas due to the physiological properties thereof

### FIELD OF THE INVENTION

The present invention relates to the process of obtaining partially purified extracts of antioxidant compounds in fruits of palm trees of the genus Euterpe. More specifically, the present invention relates to the extraction of antioxidant compounds of the fruits of the genus Euterpe palm by means of a partial and differential pulping.

### BACKGROUND OF THE INVENTION

Brazil is one of the most biologically diverse countries on the planet. Therefore, it is assigned to the term megabiodiverse. According to Conservation International, this word refers to countries with high biodiversity, which takes into account the number of endemic species—those that are unique to the region (not available elsewhere).

In this context, the search for alternatives to the use of plant resources in the Amazon becomes an extremely important factor. The development of survey research for non-timber forest products such as pharmaceuticals, cosmetics and food, is a strategy for building a model of sustainable exploitation of the biome, together with income generation.

Among the plant populations that make up this great biodiversity is the botanical genus Euterpe. Besides being a key genus in the Amazon biome, this genus Euterpe (Palm tree), offers possibilities for the market when handled and processed within the legal parameters. The fruit produced by this genus has, in its pulp, functional properties due to its high antioxidant activity related to the high content of anthocyanins and phenolic compounds.

Processes for the extraction of antioxidant compounds in palms are already well known in the art. The differentiation of extraction methods and or concentration of these antioxidant compounds are held in the type of extraction and concentration level of antioxidants obtained.

The international patent application WO 2008/156627, entitled “Phytochemical-rich oils and methods related thereto” filed on Jun. 15, 2007, on behalf of Texas A & M University System discloses a process of obtaining a lipophilic antioxidant compounds present in fruits of oil plants in the Arecaceae family, more specifically fruit species from Euterpe oleracea or Euterpe rogatory, as the agai berry the example shown in this document. The process disclosed by this document basically consists of using an extraction solution comprising an appropriate mixture of solvents from the group of alcohols (50 to 70% v: v) and acetone (30 to 50% v: v) of the mesocarp of the agai berry containing from 0.5 to 25% water. After the extraction process, the solvent mixture is removed, and the agai berry fruit oil is obtained. The oil is characterized by containing, on a dry basis, a percentage higher than 50% of unsaturated fatty acids, 10% of saturated fatty acids, 0.1% of polyphenols and 0.1% of phytosterol.

The U.S. patent U.S. Pat. No. 7,182,935, entitled “Bacterial plaque evidencing composition based on natural colorants,” filed on Jun. 19, 2002, on behalf of the Brazilian Agricultural Research Corporation—Embrapa and the Federal University of Para—UFPA describes dyes based on the agai berry (Euterpe oleracea) and annatto seeds (Bixa orellana) by means of the hydro-alcoholic extract for use as dental plaque highlighter.

The Brazilian patent application PI 0604281-3, entitled “Process for obtaining cleavage and pits of fruits of Euterpe oleracea (agai), process of obtaining hydro-alcoholic extracts from the necklines, process for obtaining freeze-dried and/or spray dryer hydro-alcoholic extract, pharmaceutical compositions containing the lyophilized and/or spray dryer of said extracts and therapeutic use of compositions as a vasodilator in the treatment of ischemic syndromes, vaso-spastic and hypertension” filed on Jul. 18, 2006, on behalf of the State University of Rio de Janeiro (UERJ) describes a process for obtaining necklines and extracts, dried or not, and pits from the fruits of Euterpe oleracea (agai berry), together with their use in pharmaceutical compositions for therapeutic use. The fruits are subjected to an extraction stage in hot water (100° C.) for 3 to 10 minutes to obtain the breaking thereof and after that the neckline was further crushed and boiled. A hydro-alcoholic extract is made from the neck by submitting it to a solid-liquid extraction at a ratio of 1:1 (v: v) in an ethanol solution, and then macerated for 10 days at room temperature or at 4° C. The hydroalcoholic extract is concentrated at low pressure and then subjected to spray drying (spray dryer) or lyophilization to obtain a dry extract with a pharmacodynamic activity for human and veterinary use.

The international patent application WO 2004/084833, entitled “Jugara and agai fruit-based dietary supplements” filed on Mar. 21, 2003, on behalf of Incorporation K2A discloses the fruit processing of a plant of the genus Euterpe to obtain a frozen or freeze-dried pulp for use in human or animal diet, by collecting, weighing, washing and pulping the fruit.

The Brazilian patent application PI 0203076-4, entitled “Process for Preparation of natural dye of agai (Euterpe oleracea, Mart-Arecaceae)” filed on May 24, 2002, on behalf of Extratos Vegetais Ativos LTDA. describes a process of preparing a natural coloring of the fruits of the agai berry (Euterpe oleracea) by selective extraction using a hydro-alcohol solution acidified directly on the fruit at room temperature. The liquid extract obtained was filtered, distilled in a vacuum and the pH adjusted to 3.5. The final product had good stability and a violet-purple color and can be used as an additive to herbal medicines, cosmetics and foods.

The Brazilian patent application PI 0107227-7, entitled “Process of acidified alcohol extraction of anthocyanins from the fruits of palm trees of the genus Euterpe,” filed on Dec. 11, 2001, on behalf of the Federal University of Para—UFPA discloses a process of obtaining an extract of anthocyanins (an antioxidant pigment) from the fruits of the palm trees of the genus Euterpe obtained directly from the hot extraction using a acidified hydro-alcoholic solution. This extract of anthocyanins produced by this method achieves half of the total concentration of this pigment as presented in the original fruit. After the extraction step, the fruit can be reprocessed so as to mitigate substantial damage to the organoleptic qualities of the final product (the agai berry). The extract produced can be used in the food industry, cosmetics, pharmaceutical and herbal and also has good storage, if kept under refrigeration.

The Brazilian patent application PI 0107103-3, entitled “Colorant and/or anthocyanin extracted from the pulp and/or the agai epicarp” filed on Oct. 16, 2001 in the name of Reynaldo Anthony Soares dos Reis describes obtaining a dye and/or anthocyanin extracted from the pulp and/or epicarp of the agai berry (Euterpe oleracea). The industrialization of the product generated therein, in the form of powder or liquid, can be used in the food or pharmaceutical areas.

As can be seen, the great disadvantage of the processes of obtaining antioxidants from fruits of palm trees as disclosed in the prior art, is that the production of antioxidant extracts is based on solutions such as hydro-alcoholic extraction solution and subsequent concentration.

### SUMMARY OF THE INVENTION

To solve the abovementioned problems, the present invention provides significant advantages over the processes for obtaining antioxidants from palm fruits (Euterpe sp.), thus allowing an increase in the performance thereof while presenting a cost/benefit ratio that is more favorable.

The present invention describes a process for extracting hydrophilic and lipophilic antioxidants from fruits of palm trees of the genus Euterpe sp. by means of a partial and differential pulping based solely in water. The extract of the present invention may or may not pass through an operation unit of adsorption on resins in order to further purify these antioxidant compounds to almost 100% on a dry basis.

The extraction of antioxidants from fruits of the genus Euterpe by means of a partial and differential pulping using only water of the present invention provides an advantage in the process for obtaining extracts, in which the extracts herein are richer in terms of antioxidants.

Additionally, the present invention describes a process for obtaining the juice from the palm fruit of the genus Euterpe, after the step of partial extraction of antioxidant compounds that is differentiated. This juice has a differential market to be standardized in the content of antioxidant compounds of either polar (hydrophilic) and nonpolar (lipophilic) nature, given the great variability of these compounds depending on ripening and/or seasonal fruits.

The extract of the present invention provides a high concentration of antioxidants, where a partial purification step can be further performed to obtain new extracts in liquid or powder form, with an even higher concentration of antioxidant compounds.

### BRIEF DESCRIPTION OF THE DRAWINGS

The structure and operation of the present invention, together with further advantages thereof, may best be understood by reference to the attached drawings and following description, which are for illustrative purposes only and not intended to limit the subject matter of the present invention.

FIG. 1 is a graph showing the kinetics of extraction of antioxidant compounds in agai berry with water using a partial and differential pulping of the present invention;

FIG. 2 is a detailed organizational chart of the process for obtaining crude extracts or purified antioxidant compounds and the juice of fruits of palm trees of the genus Euterpe the present invention.

### DETAILED DESCRIPTION OF INVENTION

The present invention relates to a process for extracting antioxidant compounds from the fruits of palm trees of the genus Euterpe by means of a partial and differential pulping using water. The process of the present invention allows one to obtain an extract with antioxidant compound concentrations ranging from 1 to almost 100% on a dry basis, in addition to producing a standardized juice of these compounds.

The steps involved in the process of the present invention for obtaining the final products are: solid-liquid extraction, phase separation (microfiltration, nanofiltration, maceration, centrifugation and pressing), concentration, adsorption and drying (lyophilization and spray drying).

The process begins by collecting the fruits of palm trees of the genus Euterpe, for example: E. oleracea, E. edulis, E. precatoria, E. espiritosantensis, among others, that depending on the level of maturity of the fruit may present very different contents of antioxidants.

These fruits have been assorted into five classes based on their maturity, according to Rogez, H; in the document entitled “Açcai: preparo, composição e melhoramento da qualidade”, Belém-PA, published by EDUFPA, 2000, incorporated herein by reference.

1—“Green” state of maturity of a set of fruits in which at least a half part thereof is still green;

2—“Vitrin”: corresponds to a set of fruit that went from green to black, the latter being in greater proportion;

3—“Black or Parau” wherein all the fruits have a black color and present a bright surface;

4—“Tuira” qualifies a black fruit partially covered by a thin film of wax, giving them a whitish appearance, and

5—“Over-ripe” wherein the fruit have the same wax, on the other hand having dry and withered characteristics.

To characterize the process of obtaining antioxidants disclosed by the present invention the class of fruits that was initially used was the one of the fruit maturity “Tuira”, but fruits classified as “Black” can also be processed. This choice is corroborated by the observed variation in fruits of the genus Euterpe palms because they had average concentrations of total anthocyanins (an antioxidant pigment) ranging from 300 to 2000 mg/kg of fruits when in these maturity levels.

To carry out the process for obtaining partially purified extracts of antioxidant compounds in fruits of palm trees of the genus Euterpe, the present invention first requires that the fruits are preserved after harvest, preferably at temperatures from 0 to 10° C., by means of a camera or chilled water, since the processing in these conditions can be extended for up to ten days. The fruit can be processed from the time of collection to fifteen hours post-harvest when kept at room temperature.

The beginning of the process is preferably treating the fruits with a water rinse, followed by a step of solid-liquid extraction (pulping 1) by means of a pulping device that can either operate in a continuous or batch mode.

The extraction of the antioxidant fraction as a proportion of antioxidants in terms of fruit mass (kg):

volume of treated water (L) from 1:1 to 1:0.25, more preferably 1:0.5.

The contact time in the extraction may vary from 1 to 150 seconds, depending on the axis of rotation of the pulping device and/or the proportion of antioxidants that intended to be extracted.

The soluble solids in the final crude aqueous extract may vary from 0.1 to 5.9%, but preferably from 0.1 to 3%, clearly indicating that most of the lumps of pulp were loosened.

After obtaining the crude aqueous extract, the fruit can be directed to two distinct processes, either to produce a standardized juice in terms of antioxidant compounds of hydrophilic character, or to the development of a standardized juice with antioxidants having both lipophilic and hydrophilic character.

In the production of standardized juice of antioxidant compounds having hydrophilic character, the fruit is directed to a second round of solid-liquid extraction (pulping 2). This process consists in plunging the fruits of the genus Euterpe in hot water (40-80° C.) for a period that may vary from 10 seconds to 4 hours so as to cause softening of the pulp and, subsequently, actually performing the pulping with potable water by means of the friction of the fruit in pulping device either in a continuous or in a batch mode.

For the production of juice comprising standard antioxidants of both lipophilic and hydrophilic character, the fruit goes through a rough pulping process for removing the mesocarp (fleshy part of the fruit), followed by a slow maceration step at a speed of 0.1 to 50 rpm at room temperature and for a maximum of four hours, but preferably for half an hour. At this stage the coalescence occurs thus leading to an oily fraction, due to different densities of the components of the fruit, being thus able to achieve a phase separation of the oily fraction (lipophilic antioxidant extract) and a non-greasy fraction.

To enhance the separation of the lipophilic extract, pressing and/or centrifugation should be performed. After obtaining the lipophilic extracts, the mesocarp rest is sent to a solid-liquid extraction (pulping 3) and standardized juices comprising both hydrophilic and lipophilic antioxidants are obtained. The said standardized juice may be characterized as partially defatted, or “light” juice, depending on the proportion of the oily fraction extracted in the previous step.

To obtain even further purified extracts of antioxidants one can use adsorption unit operation, which uses a stationary phase (macroporous resin) with high adsorptive capacity for the antioxidant compounds as described in this invention. The adsorption process can be either in batch or in fixed beds, or even by both, to raise the concentration/partial purification of the antioxidant compounds.

The use of Macroporous resin having the chemical structure of styrene-divinyl-benzene, acrylic, phenolic, or a ion exchange one can made for the process of adsorption, since they have structural affinity with the antioxidant compounds and also they all share the characteristic of being a “molecular sieve” for various constituents in crude aqueous extract (e.g., proteins and sugars).

The possible interactions between antioxidants and macroporous resins are predominantly poor. Among them, the most impressive are: hydrophobic, hydrogen bonds, van der Waals forces interactions and n-n interactions.

The stage of physical separation (centrifugation, filtration or otherwise) of the possible interfering compounds (proteins, chlorophyll, etc.) in the crude aqueous extract must be previously made, when necessary, to prevent possible loss in the adsorption capacity of the antioxidant compounds on the macroporous resin.

The resins used have the following characteristics: 1) a surface area greater than or equal to 150 m²/g; 2) a pore radius ranging from 45 to 500 angstroms (Å); 3) a pore volume from 0.05 to 3.0 mL/g; and 4) the chemical structure of: styrene-divinyl-benzene, acrylic, phenolic, or an ion exchange resin.

The crude aqueous extract generated by the pulping is conducted to an adsorption step. The adsorption of the antioxidant compounds present in the crude aqueous extract has the following characteristics: 1) pH range between 1 and 10, preferably between 1 and 4; 2) reduced content of interfering compounds such as chlorophyll and proteins; and 3) can be performed in either batch or column, or in both, preferably in a column by increasing the sorption capacity of the resin.

After the adsorption of the antioxidant compounds on the macroporous resin, it is preferable to conduct a first elution with water for possible compounds that have no affinity with the resin to elute and after that the actual removal (desorption) of the antioxidant compounds adsorbed on the resin with solvents having lower polarity when compared to water.

The desorption step of the antioxidant compounds is conducted with at least one solvent or combinations of solvents selected from the group consisting of: alcohol, aldehydes, ketones, acids and esters in concentrations ranging from 10% to 100%.

As a final step, one can concentrate these extracts obtained by means of distillation at a proper temperature, depending on the solvent used for desorption.

Another step that can be performed in the process of the present invention is a drying one, wherein the extracts are dried either by atomization (spray-dryer) or by cold drying (lyophilization) or by any other type of drying to obtain a powder that is even more concentrated in antioxidant compounds.

The use of the products obtained in the form of extract or powder, that are rich in antioxidant compounds of the present invention are of high applicability in the food industry, cosmetics, pharmaceutical and herbal since they present physiological properties.

### Preferred Embodiments of the Present Invention

The preferred embodiments of the present invention allows one to understand that the present invention should be considered an exemplification of the principles of the invention and said embodiments are not intended to limit the present invention as illustrated and described herein.

The present invention is illustrated in more detail in the following examples:

### EXAMPLE 1

Partial and differential pulping of the agai berry to obtain an aqueous extract rich in hydrophilic antioxidants

Açai berry fruits of the genus E. oleracea with less than eight hours after harvesting were washed and then submitted to a solid-liquid extraction using a batch removing device.

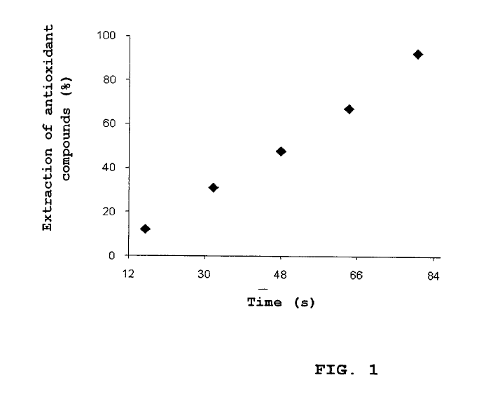

In the process of obtaining the crude aqueous extract, a mass of 5 kg of the fruit was washed with 2.5 L of water for 80 seconds. The high content of antioxidant compounds as a function of time can be observed in FIG. 1.

### EXAMPLE 2

Influence of time in the post-harvest extraction capacity of antioxidant compounds and soluble solids of the agai berry

3 kg of agai berries were used to determine the percentage of extraction of antioxidant compounds and soluble solids. A mass fraction of the fruits (1 kg) was processed at each selected time of post-harvesting (2, 24 and 48 hours). In the process of obtaining crude aqueous extract of the fruit, agai berries with 2 hours of post-harvest were left at room temperature whilst the fruits that have been processed with times of 24 and 48 hours of post-harvesting were immersed in cold water at 4° C. from the act of their collection, to ensure a high percentage of removal of antioxidant compounds without the removal of the pulp.

The process began by washing the fruits with water and then the extraction was conducted using a solid-liquid removing device in a batch mode, where a mass of 1 kg of fruit was washed with 0.5 L of water for 80 seconds.

The results of the extraction of antioxidant compounds with the soluble solids in crude aqueous extract are presented in Table 1.

TABLE 1

Amount extracted

| Time after harvest | Soluble solids | Total anthocyanins |
|---|---|---|
| (h) | (g/kg fruit) | (g/kg of fruit) |
| 2 | 0.73 | 532.36 |
| 24 | 0.91 | 522.40 |
| 48 | 1.26 | 535.11 |

The total anthocyanins are expressed as mg/kg and were obtained from the sum of cyanidin-3-glucoside and cyanidin-3-ruthinoside.

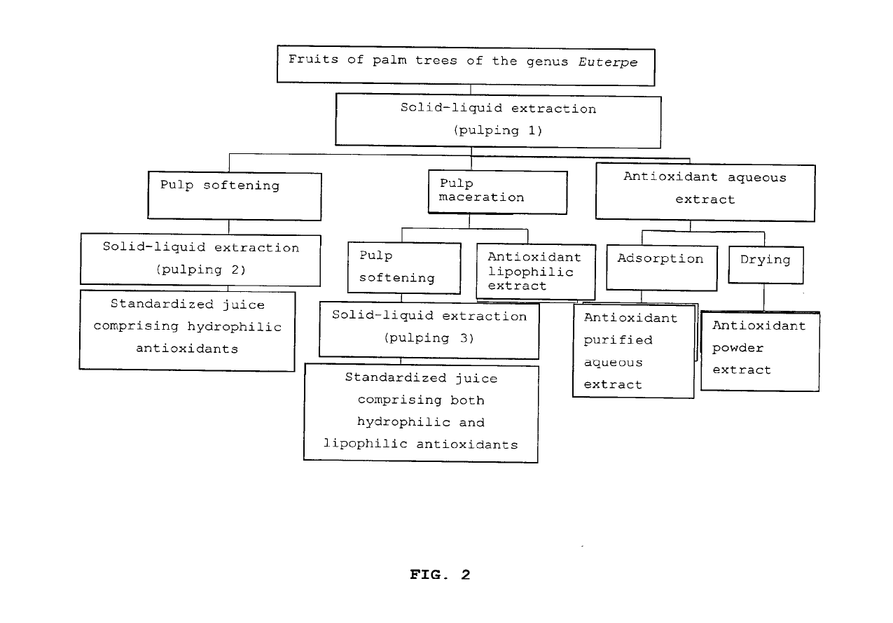

FIG. 2 shows the organizational chart detailing the process of obtaining extracts and antioxidant-rich juice standardized in both hydrophilic and lipophilic antioxidants from fruits of palm trees of the genus Euterpe.

### EXAMPLE 3

Process for obtaining lipophilic antioxidants extract from fruits of palm trees of the genus Euterpe

1.8 kg of agai berries (8 h post-harvest) were submitted to a solid-liquid extraction with 1.8 L of water. After this stage, the fruits were pulped to remove the mesocarp thereof, wherein this pulp was macerated for 30 minutes with a rotation of 10 rpm at 25° C.

The separation into two distinct phases (an oily fraction and a slightly oily fraction) was characterized by density difference. These fractions were submitted to centrifugation at 1500 rpm so as to allow said separation.

A recovery of 82% of the fraction of lipophilic antioxidants (dry basis) presented in the mesocarp has been verified.

### EXAMPLE 4

Adsorption/desorption of antioxidant compounds presented in crude aqueous extract on a acrylic macroporous resin 1 L of a crude aqueous extract of agai berries obtained from the solid-liquid extraction (pulping 1) of the agai fruits comprising a bioactive compounds (expressed as mg gallic acid equivalent/L) and total anthocyanins (expressed in mg of cyanidin-3+glucoside, cyanidin-3-ruthinosise/L) equal to 2.685 mg/L and 1.043 mg/L, respectively, was passed in an adsorption column containing 7.8 g of an acrylic resin (AR) at a rate of 50±10 ml/min at a temperature of 25° C.

After the said passing of the crude aqueous extract, 0.5 L of distilled water was added to the adsorption column in the same flow used for the extract, to remove compounds that have no affinity with the resin. After that, desorption of antioxidant compounds adsorbed on the resin was performed with 0.5 L of a 75% ethyl alcohol solution at the same flux as mentioned above.

Table 2 presents the mass balance for the adsorption of antioxidant compounds presented in the crude aqueous extract of agai berries, highlighting the amount in terms of total polyphenols and anthocyanins, and also presenting the adsorption capacity of the resin studied (RA).

TABLE 2

| | | | | Adsorption |
|---|---|---|---|---|
| | Mass in the extract (g) | | | capacity |
| Type of | | Post- | | (Mg antioxidant/ |
| antioxidant | Crude | adsorpion | Desorbed | g resin RA) |
| PTa | 2.69 | 0.31 | 1.82 | 233 |
| ANb | 1.04 | 0.1 | 0.95 | 121 |

PTa refers to the total polyphenols expressed as mg gallic acid equivalent/L of the extract and ANb refers to the total anthocyanins (cyanidin-3-glucoside+cyanidin-3-ruthinoside) in mg/L of extract.

### EXAMPLE 5

Effects of the extracts of the present invention in rich in antioxidant compound capsules for the use in the herbal industry

A group of 30 men of 41±9 years old, 13 of them having higher risk factors for the onset of coronary heart disease (total cholesterol, low density lipoproteins—LDL and thiobarbituric acid reactive substances—TBARS) was submitted to an ingestion two capsules each containing 901.6 mg of antioxidant compounds for a period 28 days.

Risk factors for the development of coronary heart diseases were evaluated during the four weeks and a significant reduction (p <0.01) was detected only in the group that already had a high risk factor, with a reduction of 4.12±1.99 mmol of malondialdehyde (MDA)/g LDL protein to 1.81±0.86 mmol MDA/g of LDL protein.

This achieved result demonstrates the functional characteristics of the extracts containing antioxidants in preventing atherosclerosis by inhibiting the peroxidation of LDL protein.

Hence, all the results herein presented clearly show the advantages of the present invention in the process of obtaining partially purified extracts of antioxidant compounds in fruits of palm trees of the genus Euterpe, wherein:
The extraction of antioxidants from fruits of palm trees of the genus Euterpe was obtained by means of a partial and differential pulping using water only;
The production of a standardized juice having an antioxidant content of either lipophilic and hydrophilic character was achieved since the process allows removing a percentage of such compounds known before the manufacturing of the juice;
The process produces extracts with high levels of purity of antioxidants, reaching almost 100% on a dry basis, which allows the use of the products obtained the form of an extract or powder, that is rich in antioxidant compounds of the present invention in the food industry, cosmetics, pharmaceutical and herbal areas.

Thus, even though there were only shown few embodiments of the present invention it will be understood by a person skilled in the art that various deletions, substitutions and changes in the process for obtaining partially purified extracts of antioxidant compounds in fruits of the genus Euterpe palms can be made without escaping from the spirit and scope of the present invention.

It is expressly stated that all combinations of elements that provide the same function in substantially the same way, to achieve the same results, are within the scope of the invention. Substitutions of elements from one mode to another are described and also fully intended and contemplated.

It should be also noted that the drawings are not necessarily drawn to scale, but rather they are only conceptual in nature. The intention herein is therefore to be solely limited, as indicated, by the scope of the appended claims.

1. Process for obtaining partially purified extracts of antioxidant compounds from fruits of palm trees of the genus Euterpe characterized in that it comprises the steps of:
a) harvesting the palm fruit;
b) preserving the said fruit at a temperature from 0° to 10° C.;
c) washing the said fruit with treated water;
d) aqueous solid-liquid extraction thereof; and
e) phase separation of the said extract.

2. Process for obtaining partially purified extracts of antioxidant compounds, according to claim 1, characterized in that the said step (a) of harvesting the fruits is applied to fruits selected according to the degree of maturity thereof.

3. Process for obtaining partially purified extracts of antioxidant compounds, according to claim 2, characterized by the fact that the fruit selected according to the maturity level thereof at step (a) is chosen according to the degree of maturity of either 3—“black” or 4—“Tuira.”

4. Process for obtaining partially purified extracts of antioxidant compounds, according to claim 1, characterized by the fact that the fruit can be processed from the time of collection to fifteen hours post-harvest, once kept at room temperature.

5. Process for obtaining partially purified extracts of antioxidant compounds, according to claim 1, characterized in that in said step (b), the preservation of the fruit is conducted by using a camera or chilled water.

6. Process for obtaining partially purified extracts of antioxidant compounds, according to claim 1, characterized by the fact that the extraction fraction is presented as a proportion of antioxidants is fruit mass (kg): volume of treated water (L) from 1:0.25 to 1:1 and in that the contact time of said extraction may vary from 1 to 150 seconds.

7. The process of obtaining partially purified extracts of antioxidant compounds, according to claim 1, characterized by the fact that the soluble solids in the antioxidant aqueous extract may vary from 0.1 to 5.9%, preferably between 0.1 and 3%, being commercially classified as clarified juice.

8. Process for obtaining partially purified extracts of antioxidant compounds, according to claim 1, characterized by the fact that the antioxidant compounds are bioactive hydrophilic and lipophilic compounds.

9. Process for obtaining partially purified extracts of antioxidant compounds, according to claim 1, characterized by the fact that the said lipophilic and hydrophilic bioactive compounds are assigned to be phenolic compounds and tocopherols.

10. Process for obtaining partially purified extracts of antioxidant compounds, according to claim 9, characterized by the fact that in the production of the standardized juice comprising antioxidants having hydrophilic character of step (a) the fruits are directed to a second round of solid-liquid extraction, called “pulping 2” step.

11. Process for obtaining partially purified extracts of antioxidant compounds, according to claim 9, characterized by the fact that the production of the standardized juice comprising antioxidant compounds of hydrophilic character in step (a) is dipping the said fruits of the genus Euterpe in a heat treatment bath between 40° C. and 80° C. for a time that may vary from 10 seconds to 4 hours.

12. Process for obtaining partially purified extracts of antioxidant compounds, according to claim 9, characterized by the fact that the softening of the pulp after the pulping 2 is conducted with drinking water by means of the friction of the fruit in a removing device in either a continuous or a batch mode.

13. Process for obtaining partially purified extracts of antioxidant compounds, according to claim 9, characterized by the fact that in the production of standard juice comprising antioxidants with both lipophilic and hydrophilic character of step (b) the fruits undergo a process of crude pulping, followed by a slow maceration at a speed from 0.1 to 50 rpm, preferably 10 rpm, at room temperature and for a maximum time of four hours, preferably of half an hour.

14. Process for obtaining partially purified extracts of antioxidant compounds, according to claim 13, characterized by the fact that the crude pulping is intended to remove the fruit.

15. Process for obtaining partially purified extracts of antioxidant compounds, according to claim 13, characterized by the fact that after the said slow maceration coalescence of oily fraction is noted.

16. Process for obtaining partially purified extracts of antioxidant compounds, according to claim 14, characterized in that the oily fraction comprises the lipophilic antioxidant extract.

17. Process for obtaining partially purified extracts of antioxidant compounds, according to claim 14, characterized by the fact that pressing and/or centrifugation is carried out so as to improve the separation of the lipophilic extract after the lipophilic extracts are obtained, the remaining mesocarp is directed to a solid-liquid extraction (pulping 3) wherein the standardized juice comprising both hydrophilic and lipophilic antioxidants is obtained.

18. Process for obtaining partially purified extracts of antioxidant compounds, according to claim 17, characterized by the fact that standardized juice comprising both hydrophilic and lipophilic antioxidants can be assigned as partially defatted or “light.”

19. Process for obtaining partially purified extracts of antioxidant compounds, according to claim 17, characterized by the fact that said standard juice comprising both hydrophilic and in lipophilic antioxidants characterization depends on the proportion of the fraction of oily antioxidant extract previously obtained.

20. Process for obtaining partially purified extracts of antioxidant compounds, according to claim 1, characterized by the fact that said separation allows to separate the antioxidant aqueous extract from organic from major organic particles through (micro-)filtration, nanofiltration, decantation, centrifugation or pressing, or different combinations of them.

21. Process for obtaining partially purified extracts of antioxidant compounds, according to claim 1, characterized by the fact that after the said separation, an enrichment in antioxidants can be performed through a) drying, or b) adsorption either in batch or in fixed beds, or in both, comprising a stationary phase adsorption using macroporous resins with high adsorptive capacity for antioxidant compounds, wherein the said macroporous resins are selected from the group consisting of styrene-divinyl-benzene resin, acrylic resin, phenolic resin or ion exchange resin.

22. Process for obtaining partially purified extracts of antioxidant compounds, according to claim 21, characterized by the fact that in the adsorption using macroporous resins according to step (b) the said antioxidant aqueous extract has a reduced content of interfering compounds, wherein said interfering compounds are selected from the group consisting of chlorophyll, proteins and sugars.

23. Process for obtaining partially purified extracts of antioxidant compounds, according to claim 21, characterized by the fact that in the adsorption using macroporous resins according to step (b) the said ion exchange resins are anionic and/or cationic having either weak and/or strong intensities.

24. Process for obtaining partially purified extracts of antioxidant compounds, according to claim 21 , characterized by the fact that in the adsorption using macroporous resins according to step (b) said process of adsorption of the antioxidant compounds present in the antioxidant aqueous extract is made between pH 1 and pH 10.

25. Process for obtaining partially purified extracts of antioxidant compounds, according to claim 21 , characterized by the fact that in the adsorption using macroporous resins according to step (b) the said process of adsorption of the antioxidant compounds presented in the antioxidant aqueous extract can be conducted in either batch, column, or both, being preferably conducted in a column present in the crude aqueous extract has a reduced content of interfering compounds, wherein said interfering compounds are selected from the group consisting of chlorophyll, proteins and sugars.

26. Process for obtaining partially purified extracts of antioxidant compounds, according to claim 21, characterized by the fact that in the adsorption using macroporous resins according to step (b) after the adsorption of the antioxidant compounds on the macroporous resin takes place, an elution with water is conducted to elute the possible compounds that have affinity with the resin, followed by the removal process by desorption of antioxidant compounds adsorbed on the resin with solvents of lower polarity when compared to water.

27. Process for obtaining partially purified extracts of antioxidant compounds, according to claim 25, characterized in that the said step of desorption of antioxidant compounds is conducted with at least one solvent or with combinations of solvents selected from the group consisting of alcohol, aldehydes, ketones, acids and esters in concentrations ranging from 10% to 100%.

28. Process for obtaining partially purified extracts of antioxidant compounds, according to claim 1, characterized by the fact that the palm trees of the genus Euterpe comprise selected species of E. oleracea, E. edulis, E. precatoria and E. espiritosantensis.

29. Process for obtaining partially purified extracts of antioxidant compounds, according to claim 1, characterized by the fact that the proportion of antioxidants extracted from fruits by partial and differential pulping ranges from 0.1 to 100%.

30. Process for obtaining partially purified extracts of antioxidant compounds, according to claim 1, characterized in that the step of aqueous solid-liquid extraction is called “pulping 1,” wherein said pulping removing device can occur in either a continuous or a batch mode.

31. Process for obtaining partially purified extracts of antioxidant compounds, according to claim 1, characterized by the fact that after obtaining the said antioxidant aqueous extract, the fruit can be lead to two distinct processes:
a) to produce a standardized juice comprising antioxidant compounds of hydrophilic character, or
b) to prepare a standardized juice comprising antioxidants of both lipophilic and hydrophilic character, and an oily antioxidant extract.

32. Process for obtaining partially purified extracts of antioxidant compounds, according to claim 14, characterized by the fact that the phase separation of the oily fraction from the slightly oily fraction is due to the density difference of the components of the fruit.

33. Process for obtaining partially purified extracts of antioxidant compounds, according to claim 14, characterized by the fact that after the lipophilic extracts are obtained, the remaining mesocarp is directed to a solid-liquid extraction (pulping 3) wherein the standardized juice comprising both hydrophilic and lipophilic antioxidants is obtained.

34. Process for obtaining partially purified extracts of antioxidant compounds, according to claim 21, characterized by the fact that in the adsorption using macroporous resins according to step (b) the said macroporous resins have a surface area greater than or equal to 150 m²/g, a pore radius ranging from 45 to 500 angstroms (Å) and a pore volume from 0.05 to 3.0 mL/g.

35. Process for obtaining partially purified extracts of antioxidant compounds, according to claim 1, characterized by the fact that the extracts can be concentrated by distillation at a proper temperature, depending on the solvent used for desorption.
